(19) United States
(12) Patent Application Publication
(10) Pub. No.: US 2015/0223735 A1
(43) Pub. Date: Aug. 13, 2015

(54) Antigen Monitoring System

(71) Applicant: Marlon Williams Williams, Boynton Beach, FL (US)

(72) Inventor: Marlon Williams Williams, Boynton Beach, FL (US)

(21) Appl. No.: 14/177,181

(22) Filed: Feb. 10, 2014

(51) Int. Cl. A61B 5/1455; A61B 5/00; A61K 49/00; A61B 5/145

(52) U.S. Cl. CPC A61B 5/1455; A61B 5/14546; A61B 5/0022; A61B 5/7278; A61K 49/00

(57) ABSTRACT

A method for detecting cancer levels in a human patient by monitoring antigen levels in the blood stream of the patient. Polysilicon mirrors are administrated into the blood stream of the b patient orally or by injection. Near infrared light is transmitted through the patient's skin and reflected by the mirrors to a detector that converts the light to a readable signal for calculating the cancer level based on the level of antigen markers in the patients blood stream.

### RELATED PATENTS

The present application relates to applicant's U.S. Pat. No. 8,463,344 the disclosure of which is incorporated herein by reference.

### FIELD OF THE INVENTION

The present invention relates to a method of preparing porous silicon mirrors for use in wireless monitoring of antigen levels in the blood, particularly in the blood of cancer patients.

### BACKGROUND OF THE INVENTION

Certain types of cancer are associated with antigen markers in the blood. These markers provide a potential for early diagnosis by detection and monitoring of these antigen markers. Examples of cancers include, but are not limited to, colorectal cancer, lung cancers, prostate cancer, pancreatic cancer, ovarian and uterine cancers and breast cancer.

While cancer is generally more receptive to treatment if diagnosed in the early stages, it can be difficult to detect in an early stage. With the recent emergence of genetic expression profiling, oncologists have broken down malignancies to their genetic profile which will allow them to classify cancers into distinct categories. Tissues sampled for such genetic expression profiling can be studied for antigen markers associated with additional types of cancers.

Existing methods of detecting and monitoring cancers are time consuming and complex. A patient is required to go to a testing facility such as a doctor's office or lab and have blood drawn. The blood must then be tested in a laboratory, either on-site or sent to a remote location, to determine the level of antigen markers for many different types of cancers. Costly equipment is required and the process takes a great deal of time. This is particularly true when a patient has to be tested on a regular basis such as weekly or monthly. In addition drawing blood often creates discomfort as well as being an invasive procedure.

A biomaterial is a non-living material used in a medical device which is intended to interact with biological systems. Such materials may be relatively “bioinert”, “biocompatible”, “bioactive” or “unresorbable” depending upon their biological response in vivo.

When silicon is deliberately riddled with nanometer sized holes, it becomes biocompatible and will not be rejected by the body and is biodegradable and will dissolve harmlessly over time.

Porous silicon does not need to be shielded from bodily tissue and the blood stream. The only byproduct is silicic acid, a commonly used substance. Therefore porous silicon can be used in a variety medical uses as referred to in the parent application.

Prior patent art of interest includes the US patent Application No. 2004/0078219 to Kaylor et al, that relates to a measurement arrangement including a biosensor using silicon based mirrors in communication with a personal control means and processors. Cancer markers bind to the mirrors to change the reflectivity. Kaylor et al uses a bio-implanted or implantable sensor and provides no teaching of administering the mirrors randomly in the patient's blood stream orally or by injection. Rather the mirrors disclosed in Kaylor et al are inoperable if used orally or by injection.

Another patent of interest is U.S. Pat. No. 6,774,209 to Rondon et al that teaches the use of binding peptides with cancer markers.

Still another patent of interest is U.S. Pat. No. 5,571,152 to Chen et al that uses localized implantable circuits within silicone beads that are injected into disease sites. There is no teaching of injecting randomly into a patient's bloodstream.

Other patents and applications that show the state of the art include US patent application No. 2005/0266045 to Canham et al, and U.S. Pat. No. 6,208,894 to Schulman et al, that teaches the use of injecting into the body miniature microbe processors and U.S. Pat. No. 6,398,710 to Ishikawa that monitors radiation received during radiation therapy.

### SUMMARY OF THE PRESENT INVENTION

The present invention is a method of non-invasive detecting or monitoring cancer levels in a patient. The current application relates to Applicant's U.S. Pat. No. 8,463,344 which is incorporated herein by way of reference. Porous silicon mirrors are treated to bond with cancer antigen markers and are monitored to provide an indication of cancer levels in the patient. This is accomplished by administering the light reflecting porous silicon mirror material randomly into the blood stream of the patient.

The antigen levels are monitored by a light source that is reflected through the patient's skin. A near infrared light source is a preferred reflective light source for use with porous silicon mirrors that are present in the patient's blood stream. Light reflected from the light reflecting, porous silicon mirrors through the skin of the patient is received by a suitable light detecting sensor and is converted into a readable signal, preferably a digital signal that is used to calculate the amount of antigen marker levels in the patient's blood stream. Preferably the measurements are then compared with previously taken and stored measurements to determine the current state of the patient's cancer level. The measurements may be used to activate a patient held device or to send a signal to a remote monitoring location.

The method of the present invention can be practiced by administering the reflective material orally or by injection as long as they assume random locations throughout the patient's blood stream.

Once the measurements are taken and compared, the results may be transmitted to a remote location by conventional communication systems such as the Internet.

The method preferably is practiced by using a near infrared light source and detector for transmitting light through the patient's skin such that it is reflected by the porous silicon mirrors and detected by the near infrared source to create an analog signal based on the strength of the reflected light. The analog signal is transmitted to an analog to digital converter to obtain a digital signal of the reflected light. The digital signal is transmitted to a microcontroller where it is stored in a memory device where it may be analyzed or transmitted further through a suitable communication system for further monitoring and/or comparison with previously stored data. By measuring the antigen markers in a patient's blood over a period of time the level of cancer can be used to determine a protocol for the particular patient.

Summarizing the present invention, the method calls for administering orally or by injection of a biomedical form of pre-treated elemental silicon that serves as a mirror surface to reflect light, such as near infrared light, that is transmitted through the patient's skin. The porous silicon mirrors are located randomly in the bloodstream and bind to antigen markers at the disease site. The porous silicon mirrors may be externally accessed at any convenient location through the skin on the body and do not require monitoring at the disease site. Binding of the antigen markers to the mirrors reduces the reflectivity of the mirrors. The resultant signal is processed by appropriate electronic circuitry and may be used for comparison with previously stored measurements to monitor the patient's progress with the disease.

Among the objects of the present invention is the provision of a method for detecting the cancer level of a patient.

Another object is the provision of a method of detecting the antigen marker levels in a patient's blood stream and using the results to determine cancer levels.

Still another object is the provision of a method of determining the cancer levels in a patient that is non-invasive and is capable of immediate processing without addition testing.

Yet another object is the provision of a cancer detecting/monitoring method that does not require access to the disease site

These and other objects may be determined by reference to the following specification and accompanying drawing.

### DESCRIPTION OF THE DRAWINGS

FIG. 1 illustrates a cancer antigen detection system used in the method of the present invention.

### DESCRIPTION OF THE PREFERRED EMBODIMENTS

The present invention is a method for detecting the presence cancer in patients by determining the cancer antigen marker levels. The method includes administering reflective material in the form of porous silicon mirrors randomly into a patient's bloodstream, transmitting light through the patient's skin onto the reflective material and reading the results to determine the antigen markers of various cancers to thereby determine the cancer levels. A preferable method uses porous silicon mirrors as reflectors and a near infrared light transmitter/sensor as the source of reflected light. An analog to digital converter measures the intensity of the reflected light and converts it to a digital signal that is fed to a microcontroller and suitable display and/or monitoring apparatus.

Silicon mirrors are derivatized by a chemical technique which transforms the chemical compound into a product that is the derivate of the reaction that has similar chemical structure. An initial step in the method of the present invention is bioconjugating a supply of derivatized porous silicon mirrors. Bioconjugation is a process of coupling two biomolecules together in a covalent linkage. An example is the coupling of an antibody to an enzyme. In the present invention an immunoassay is bioconjugated onto the surface of porous silicon mirrors by replacing the hydrogen in the mirrors with a functional organic group that can be bound to the desired protein molecule of the cancer antigen.

The porous silicon mirrors are formed by silicon being riddled with nanometer-sized holes. Once the silicon becomes porous, it acts as a highly reflective mirror. The holes are created using pulsed anodic etching in a HF electrolyte solution. A preferable method uses the electrolyte solution in a cylindrical PVC electrochemical beaker with a platinum cathode sheet and crystalline silicon wafers as the anode.

The etching solution is made up of three parts hydrofluoric acid and one part anhydrous ethanol. The silicon wafers are etched to a depth of 100 nm to 1000 nm. The thickness of the resulting mirror is determined by selectively removing the porous layer in a solution of potassium hydroxide. After forming the selected depth of the layers, the mirrors are rinsed with deionized water and ethanol and blown dry with nitrogen gas.

Preparation of the mirrors creates a covalent bond between the cancer antibody, the immunoassay, and the porous silicon surface. The amount of cancer antibody that is attached to the porous silicon surface determines the reflectivity level of the surface. The greater the antibody level, the less reflective the mirrors become and this level can be measured as described hereinbelow.

In order to create the covalent bond the porous silicon is placed in a quartz cuvette with a 10% volume solution of monomeric acrylic acid in ethanol. The solution is de-aerated using oxygen free flux to prevent photo generated radical reaction inhibition. The porous silicon is then exposed to 350 nm of UV light for approximately one hour.

This procedure replaces the silicon surface hydrogen termination. Next, a diazirine/succinimidyl b-linker is used to bind an immunoassay to the terminated porous silicon surface that was activated by the cross link succinimidyl. After the cross linking reaction, the activated porous silicon is washed with tetrachloride and immediately immersed in a solution containing the cancer immunoassay.

Once the silicon mirrors are prepared, they are administered in vivo randomly in a patient's bloodstream. This treatment also allows the silicon to become biocompatible, whereby the body does not reject the silicon, and subsequently becomes biodegradable by dissolving in the bloodstream harmlessly over a period of time. The porous silicon mirrors may be administered orally in a capsule swallowed by the patient. Alternately the porous silicon mirrors may be injected into the bloodstream or implanted under the skin.

The major differences between the oral and injection methods of administering the silicon mirrors, is the absorption time. The injected mirrors will reach a maximum absorption of the antigen quicker. The orally taken mirrors have to pass through the digestive system including the acidic environment of the stomach before entering the blood stream. Preferably the porous silicon mirrors are taken on an empty stomach because food can delay absorption. Preferably the degradation/absorption rate is approximately 12 hours as set upon the porous silicon mirrors in the lab. This results in a large portion of them, as much as 85%, being diffused directly into the bloodstream via the stomach wall. The remaining 15% will pass through the pyloric junction into the small intestine where they encounter the acidic environment created by the digestive material of the lower stomach. There the porous silicon mirrors are absorbed by the villi and transported to the bloodstream in much the same way that nutrients from solid food are removed and used by the body for nutritional purposes. The 12 hour degradation/absorption rate allows maximum degradation and ample time for the covalent bond to form between the antigen markers and the porous silicon mirrors in order to give a positive reading during subsequent monitoring as described above. Continued monitoring requires the patient to take additional porous silicon mirrors at 12 hour intervals in order to provide a continuous monitoring process.

The poly silicon mirrors may also be implanted under the skin next to a blood vessel or injected directly into the blood vessel. When injected, the covalent bonding process of the antigen markers and the porous silicon mirrors is immediate and the degradation time is doubled by eliminating the effects of the digestion process. However the injection method is more invasive and requires injection every 24 hours.

The porous silicon mirrors are treated with particular immunoassays that relate to the particular cancer being monitored by insertion into the etchings in the silicon surface. The antigen markers in the patient's blood bind to the porous silicon mirrors and represent the cancer levels in the patient.

Once the porous silicon mirrors are present in a patient's bloodstream, non-evasive measurements can be made at any suitable location on a patient's body to determine the level of antigen markers present. This is accomplished with a suitable light source through the skin of the patient and associated electronic circuitry and components that measure the reflected light from the porous silicon mirrors.

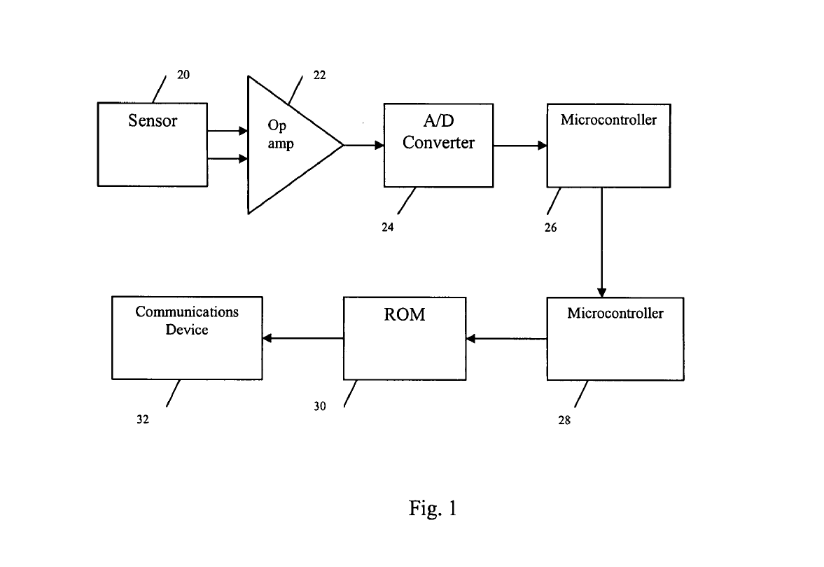

Referring to FIG. 1, a suitable embodiment of a circuitry system includes a sensor 20 such as a retroreflective photoelectric sensor that measures reflected light to determine the properties of an object being measured. The sensor 20 includes a light transmitter and a photoelectric receiver. In the present embodiment the light transmitter is a near infrared light source although it will be appreciated other light sources may be used in keeping within the broader aspects of the present method.

The output of the sensor 20 is fed to an amplifier 22 that, in turn, is fed to an analog to digital converter 24 that converts the analog signal to a digital format. A proportional relationship between antigen concentration on the porous silicon mirrors and the intensity of the signal is established whereby a higher level of antigen reduces the reflectivity of the light and thus the signal level. The resulting digital signal is received by microcontrollers 26 and 28 that produce a representation of the antigen level in a patient's bloodstream. A read only memory, ROM, 30 stores the data that is subsequently sent to a communication device 32 that is capable of wireless transmission of the measured antigen level to an interested party, for example, the patient's doctor or similar monitoring venue. The information may be used in a variety of ways. The electronic system, including the components of the system and the function thereof, is described in detail in the parent U.S. Pat. No. 8,463,344 referenced hereinabove.

A specific example of the method of the present invention is used to determine colorectal cancer in a patient by monitoring the carcinoembryonic antigen, hereinafter CEA, level in the patient's bloodstream. CEA is a lycoprotein involved in cell adhesion and is not present in healthy adults. It is present in human colon cancer extracts and measurement of the CEA levels is representative of the presence of and/or the amount of cancer in the body. After administering porous silicon mirrors with CEA-specific immunoassays into the patient's bloodstream, orally or by injection, the CEA antigen molecules bind to the mirrors in accordance with the specific cancer level in the patient′ system. Non-invasive detection and monitoring of the antigen levels is accomplished using a near infrared light source that emits light through the patient's skin at a select location, such as a finger, that may be remote from the area of cancer in the body. The reflectivity of the porous silicon mirrors is lessened by the CEA attached thereto and the resultant light signal provides a digital measurement of the antigen markers.

In addition to detecting and monitoring colorectal cancer, it is understood that the present invention contemplates detecting and monitoring a wide variety of antigen markers of other cancers such as, but not limited to, ovarian, cervix and uterus, testicular, gastrointestinal, pancreatic, and lung cancers as examples.

The scope of the present invention is not limited by the above description but be determined with reference to the following claims.

1. A method for monitoring an antigen level in a human subject, comprising:
administering a multiplicity of photo reflective elements to said human subject, said photo reflective elements positioned randomly in the bloodstream of said human subject;
activating a representative number of said photo-reflective elements by placing an area of skin of the patient on a light-emitting sensor;
detecting light reflected through the skin of the subject from said photo-reflective elements; and
calculating an antigen level in the subject's blood stream based upon a level of light detected from said photo-reflective elements.

2. The method of claim 1, wherein the intensity of light reflected from said photo-reflective elements varies according to the amount of antigen markers in the subject's blood.

3. A method for monitoring an antigen level in a human subject, comprising:
bio-conjugating a supply of derivatized photo-reflective, porous silicon mirrors;
administering a multiplicity of said silicon mirrors to said human subject, said silicon mirrors positioned randomly in the bloodstream of said human subject;
activating a representative number of said photo-reflective mirrors by placing an area of skin of the patient on a light-emitting sensor;
detecting light reflected through the skin of the subject from said photo-reflective mirrors; and
calculating an antigen level in the subject's blood stream based upon a level of light detected from said photo-reflective elements.

4. The method of claim 3 wherein said bio-conjugating step is defined by replacing the hydrogen with a functional organic group capable of bonding to a cancer antigen molecule.
The method of claim 3 wherein said bonding step is achieved using a cross link.

5. The method of claim 3 wherein said porous silicon mirrors are prepared using a pulsed etching in an HF electrolyte solution.

6. The method of claim 3 wherein said administering step is further defined as oral administration using a capsule like container for the porous silicon mirrors.

7. The method of claim 3 wherein said administering step is further defined as an injection into the bloodstream of said subject.

8. The method of claim 1 wherein said photo-reflective elements are defined as poly-silicon mirrors.

9. The method of claim 2 wherein said poly-silicon mirrors are biodegradable.

10. The method of claim 2 wherein said light emitting sensor is a retro-reflective photoelectric sensor.

11. The method of claim 2 wherein light from said light sensor is near infrared.

12. A method for detecting or monitoring cancer in a subject, comprising:
administering polysilicon mirrors to random locations within the bloodstream of a subject orally or by injection;
transmitting near infrared light through the subject's skin;
detecting light which is reflected from the polysilicon mirrors in the blood stream though the subject's skin;
converting received light into a digital signal; and
calculating an antigen level in the subject's blood from the digital signal.

13. The method of claim 12 wherein differences in the antigen level in a subject's blood are determined based on previously stored measurements compared with currently taken measurements.

14. The method of claim 12 wherein the level of CEA is transmitted to a third party.

15. The method of claim 12 wherein the level of CEA is transmitted to the third party via an Internet.
